(19) United States
(12) Patent Application Publication
(10) Pub. No.: US 2024/0165062 A1
(43) Pub. Date: May 23, 2024

(54) COMPOSITION FOR INCREASING MUSCLE

(71) Applicants: YUKI GOSEI KOGYO CO., LTD., Tokyo (JP); KYUSHU UNIVERSITY, NATIONAL UNIVERSITY CORPORATION, Fukuoka-shi (JP)

(72) Inventors: Tetsuji NODA, Tokyo (JP); Yoshinori KATAKURA, Fukuoka-shi (JP)

(73) Assignees: YUKI GOSEI KOGYO CO., LTD., Tokyo (JP); KYUSHU UNIVERSITY, NATIONAL UNIVERSITY CORPORATION, Fukuoka-shi (JP)

(21) Appl. No.: 18/456,744

(22) Filed: Aug. 28, 2023

(30) Foreign Application Priority Data
Aug. 30, 2022 (JP) 2022137310

(51) Int. Cl. A61K 31/197; A61P 21/00

(52) U.S. Cl. CPC A61K 31/197; A61P 21/00

(57) ABSTRACT

[OBJECT]

The purpose of this invention is to provide a muscle-increasing ingredient with excellent safety.

[MEANS FOR SOLUTION]

The problem can be solved by a composition for increasing muscle, comprising β-alanine or a salt thereof as an active ingredient of the present invention of the present invention.

### TECHNICAL FIELD

The present invention relates to a composition for increasing muscle.

### BACKGROUND ART

Skeletal muscle tissues are classified into slow muscle type and fast muscle type based on differences in metabolism and contractile properties. The slow muscle type contracts slowly and continuously, and an increase of skeletal muscles of slow muscle type is effective in improving a lack of physical exercise. The fast muscle type contracts at a high rate and exert great force instantaneously, but it is said that their number is decreased with age. Increasing fast muscles enables instantaneous movements and prevents hurts.

On the other hand, from the viewpoint of animal meat, it has been reported in a study using pigs that increased muscle fibers of slow muscle type increase taurine, carnitine, and iron, and further improve softness and juiciness (Non-patent literature 1).

For example, an agent for increasing slow muscle containing oleic acid has been reported as an agent for increasing skeletal muscles of slow muscle type (Patent literature 1). In addition, an agent for increasing fast muscle containing juniper berry extract and the like has been reported as an agent for increasing skeletal muscles of fast muscle type (Patent literature 2).

### CITATION LIST

### Patent Literature

[Patent literature 1] JP 2021-170955 A
[Patent literature 2] JP 2013-100272 A

### Non-Patent Literature

[Non-patent literature 1] Kang Y. K. et al., Effects of myosin heavy chain isoforms on meat quality, fatty acid composition, and sensory evaluation in Berkshire pigs, Meat Science, 89, 384-389 (2011)

### SUMMARY OF INVENTION

### Technical Problem

However, the development of muscle-increasing ingredients with excellent safety is expected. Therefore, the purpose of this invention is to provide a muscle-increasing ingredient with excellent safety.

### Solution to Problem

The present inventors have conducted intensive studies into a muscle-increasing ingredient with excellent safety, as a result, surprisingly found that β-alanine exhibits an excellent muscle-increasing effect.

The present invention is based on the above findings.

Accordingly, the present invention relates to:
[1] a composition for increasing muscle, comprising β-alanine or a salt thereof as an active ingredient,
[2] the composition for increasing muscle of the item [1], wherein the muscle is a slow muscle or a fast muscle,
[3] the composition for increasing muscle of the item [2], wherein muscle gain is a slow muscle gain or a fast muscle gain at differentiation of myoblasts into myotube cells, and
[4] the composition for increasing muscle of the item [2] or [3], wherein the composition is food composition.

Further, the present specification discloses:
[5] a method for increasing muscle, comprising a step of administrating an effective amount of β-alanine or a salt thereof to a subject (the method may be conducted as a medical practice, or a medical practice may be excluded from the method), and
[6] the method for increasing muscle of the item [5], wherein the muscle is a slow muscle or a fast muscle (the method may be conducted as a medical practice, or a medical practice may be excluded from the method).

### Advantageous Effects of Invention

According to the composition for increasing muscle of the present invention, the slow muscle and fast muscle can be effectively increased. In addition, it is possible to increase slow muscle and fast muscle in meat such as cattle and pigs.

### BRIEF DESCRIPTION OF THE DRAWINGS

FIG. 1 is graphs showing expressions of MyHCI, MyHCIIa, MyHCIIb, and MyHCIIx genes in C2C12 cells by addition of β-alanine.

FIG. 2 is a graph showing expression of MyoD gene in C2C12 cells by addition of 3-alanine.

FIG. 3 is a fluorescence micrographs showing muscle cells differentiated from myoblasts to myotube cells, and graphs showing the ratio of slow muscle cells to fast muscle cells, by addition of β-alanine.

FIG. 4 is graphs showing the mRNA expressions of MyHCI (slow muscle)(A), MyHCIIa (fast muscle)(B), MyHCIIx (fast muscle)(C), and MyHCIIb (fast muscle) (D) measured by RT-qPCR in the C2C12 cells wherein the differentiated C2C12 cells were then treated by addition of 0.5, 1, or 10 (mM) of β-alanine for 2 days.

### DESCRIPTION OF EMBODIMENTS

The composition for increasing muscle of the present invention comprises β-alanine or salt thereof as an active ingredient.

β-alanine is represented by the following formula [1]:

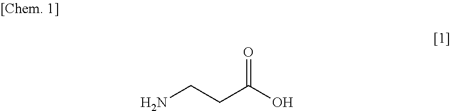

and is also called 3-aminopropanoic acid. Extracts, concentrates, or purified products from foods or natural products that contain relatively large amounts of β-alanine, can be used, as 3-alanine comprised in the composition for increasing muscle of the present invention. In addition, synthetic β-alanine may be used. For example, β-alanine can be synthesized from 3-propiolactone by the β-alanine synthesis method (Ford, Org. Sys. Coll. Vol. 3, 34(1955)). As another synthetic method, it can be synthesized from acrylonitrile and ammonia. The composition for increasing muscle of the present invention can comprise β-alanine as its salt, hydrate, or solvate.

The salt of β-alanine is not limited, so long as it is a salt with an inorganic base or an organic base, or a salt with an acid, and is a salt acceptable for medicine food, or cosmetic. Specific examples of the salt with the inorganic base or the organic base include a salt with an inorganic base, an organic base, or a metallic alkoxide. They can be prepared by mixing 3-alanine with an inorganic base, an organic base, or a metallic alkoxide.

As the inorganic bases that can form salts, there may be mentioned a hydroxide, carbonate, hydrogen carbonate, acetate, or hydride of alkali metals (such as lithium, sodium, potassium, or the like); a hydroxide, hydride, or the like of alkaline earth metals (such as magnesium, calcium, or barium). As the organic bases that can form salts, there may be mentioned dimethylamine, triethylamine, piperazine, pyrrolidine, piperidine, 2-phenylethylamine, benzylamine, ethanolamine, diethanolamine, pyridine, collidine, or the like. Further, as the metallic alkoxide, there may be mentioned sodium methoxide, potassium tert-butoxide, magnesium methoxide, or the like. The salt of β-alanine is preferably a sodium salt, potassium salt, calcium salt, or a combination thereof.

Specific examples of the salt with an acid include a salt with an inorganic acid or an organic acid. As the inorganic acid that can form a salt, there may be mentioned hydrochloric acid.

### <<Genes Related to Slow Muscle and Fast Muscle>>

MyHCI is a slow muscle marker, and MyHCIIa, MyHCIIb, and MyHCIIx are fast muscle markers. Further, MyoD is a muscle differentiation transcriptional regulator that promotes the differentiation of myoblasts into myotube cells.

By increasing the expression of MyHCI, it is possible to promote the differentiation of myoblasts into slow muscle cells and increase the number of slow muscle cells. By increasing the expression of MyHCIIa, MyHCIIb, and MyHCIIx, it is possible to promote the differentiation of myoblasts into fast muscle cells and increase the number of fast muscle cells.

### <<Slow Muscle Fiber and Fast Muscle Fiber>>

Muscle fibers (myocytes) that form skeletal muscles are multinucleated cells, and a group of myofibers constitutes a muscle bundle, and a group of muscle bundles constitutes a skeletal muscle. The skeletal muscles are classified into slow muscle fibers (sometimes referred to as slow muscle) and fast muscle fibers (sometimes referred to as fast muscle). The slow muscle fibers are generally red in color. Red is the color of oxygen-binding myoglobin and the slow muscle fiber is referred to as Type 1. On the other hand, the fast muscle fibers are white in color and are referred to as Type2. The composition for increasing muscle can increase the above-mentioned slow muscle fiber and/or fast muscle fiber.

<<Differentiation into Slow Muscle Cells>>

β-alanine used in the present invention is not limited, but can promote the increase of slow muscle during differentiation. When myoblasts differentiate into myotube cells, they differentiate into slow muscle cells or fast muscle cells. The composition for increasing muscle of the present invention can increase the number of slow muscle cells that differentiate from myoblasts.

<<Differentiation into Fast Muscle Cells>>

β-alanine used in the present invention is not limited, but can promote the increase of fast muscle during differentiation of myoblasts into myotube cells.

When myoblasts differentiate into myotube cells, they differentiate into slow muscle cells or fast muscle cells. The composition for increasing muscle of the present invention can increase the number of fast muscle cells that differentiate from myoblasts.

The formulation of the composition for increasing muscle of the present invention is not particularly limited. For example, oral agents, such as powders, subtle granules, granules, tablets, capsules, suspensions, emulsions, syrups, extracts, or balls; or parenteral agents, such as injections, liquid for external use, ointments, suppositories, creams for local administration, or eye-drops, there can be mentioned.

The above oral agent can be prepared in accordance with conventional methods, using excipients, such as gelatin, alginate sodium, starch, cornstarch, saccharose, lactose, glucose, mannitol, carboxymethyl-cellulose, dextrin, polyvinyl pyrrolidone, crystalline cellulose, soy lecithin, sucrose, fatty acid ester, talc, magnesium stearate, polyethylene glycol, magnesium silicate, silicic anhydride, or synthetic aluminum silicate; binders, disintegrators, surfactants, lubricants, flow accelerator, diluents, preservatives, colorants, flavors, corrigents, stabilizers, humectants, antiseptics, antioxidant, or the like.

Examples of the parenteral agents include injections. In a preparation of the injections, an aqueous solvent such as normal saline solution or Ringer solution, non-aqueous solutions such as plant oil or fatty acid ester, a tonicity agent such as glucose or sodium chloride, a solubility assisting agent, a stabilizing agent, an antiseptic agent, a suspending agent, or an emulsifying agent, can be optionally used, in addition to the active ingredient.

A dose of the composition for increasing muscle may be appropriately determined in accordance with, for example, age, sex, body weight, or degree of symptom of each patient, route of administration, or the like, and the determined dosage can be administered orally or parenterally. For example, in the case of an adult, the intake amount of the composition for increasing muscle of the present invention is preferably 0.01 to 100 mg/kg per day as β-alanine. The above administration method is an example, and other administration methods may be used. It is desirable that the administration method, dose, administration period, administration interval, and the like, of the composition for increasing muscle to humans are determined by a controlled clinical trial.

In addition, the dosage form is not limited to a drug medicine, but can be administered as a food composition (for example, functional food, health food, or beverage) or animal feed composition as described below. The compositions for increasing muscle may be used for healthy subjects or subjects with some diseases.

As a method for manufacturing the composition for increasing muscle containing (3-alanine, known methods for manufacturing pharmaceutical composition, food composition, or animal feed compositions can be used, except that β-alanine is contained as an active ingredient.

The composition for increasing muscle of the present invention may contain other components. Examples of the other components include, for example, emulsifiers such as edible fats and oils, water, glycerin fatty acid ester, sucrose fatty acid ester, sorbitan fatty acid ester, propylene glycol fatty acid ester, glycerin organic acid fatty acid ester, polyglycerol fatty acid ester, calcium stearoyl lactylate, sodium stearoyl lactate, polyoxyethylene sorbitan fatty acid ester; thickening stabilizers such as locust bean gum, carrageenan, alginic acids, pectin, xanthan gum, crystalline cellulose, carboxymethyl cellulose, methyl cellulose, agar, glucomannan, gelatin, starch, or chemical starch; salty taste agents such as salt, or potassium chloride; acidulants such as acetic acid, lactic acid, or gluconic acid; sugars or sugar alcohols; sweeteners such as stevia or aspartame; colorants such as beta-carotene, caramel, or red koji pigment; antioxidants such as tocopherol or tea extract; food materials or food additives such as flavoring agent; pH adjuster; food preservative, or shelf life improver. Further, the composition for increasing muscle may contain various vitamins, or functional materials such as coenzyme Q, plant sterol, or milk fat globule membrane. The amount of these other components is preferably 80% by mass or less, more preferably 40% by mass or less, and further preferably 20% by mass or less, as a total amount in the composition for increasing muscles of the present invention.

### <<Food Composition>>

The composition for increasing muscle of the present invention may be a food composition. The food compositions for increasing muscle of the present invention comprises β-alanine or a salt thereof. The food composition for increasing muscle of the present invention is not limited, as long as it can be administered orally.

The food in the food composition for increasing muscle of the present invention is a food or drink, and includes a beverage. The food in the present invention is not particularly limited, for example, there may be mentioned seasonings such as miso, soy sauce, sauce for noodles, sauce, soup stock, pasta sauce, dressing, mayonnaise, tomato ketchup, Worcestershire sauce, sauce for pork cutlet, or sprinkle; instant cooked foods such as a soup base, curry roux, white sauce, rice with tea base, or soup base; soups such as miso soup, soup, consomme soup, or potage soup; processed livestock products such as grilled meat, ham, or sausage; a meat alternative (fake meat) manufactured from soybeans, peas, and the like; processed marine products such as boiled fish paste, dried fish, salted fish guts, fish boiled in soy sauce, rare delicacy; processed vegetable products such as pickles; snacks such as potato chips, or rice cracker; bakery foods such as bread, sweet bread, or cookies; cooked foods such as boiled foods, fried foods, grilled foods, curry, stew, gratin, rice, porridge, or rice ball; noodles such as pasta, wheat noodle, or ramen; fat processed foods such as margarine, shortening, fat spread, or flavored fat spread; materials for confectionery and bread such as flower pastes, or bean paste; mixed powders such as bread mix powder, cake mix powder, or fried food mix powder; confectioneries such as chocolate, candy, jelly, ice cream, or gum; Japanese confectioneries such as steamed bun, or castella; beverages such as coffee, coffee milk, tea, milk tea, soy milk, nutritional drink, vegetable drink, vinegared drink, juice, cola, mineral water, or sports drink; alcoholic beverages such as beer, wine, cocktail, or sour; milk and dairy products such as bovine milk, yogurt, or cheese.

The food composition for increasing muscle of the present invention can be prepared by using the known methods for manufacturing foods and drinks except for comprising 3-alanine.

### <<Animal Feed Composition>>

The composition for increasing muscle of the present invention can be used as an animal feed composition. The feed is not limited, but includes feed for industrial animals or feed for pets (pet food).

Animals include all non-ruminants and ruminants. As the non-ruminants, there may be mentioned horse, pig, poultry (such as turkey, duck, chicken, broiler, layer), fish (such as salmon, trout, tilapia, catfish, and carp), and crustaceans (such as shrimp). As the non-ruminants, there may be mentioned pet such as dog, cat, rabbit, hamster, guinea pig, and squirrel; and laboratory animal such as mouse and rat. As the ruminants, there may be mentioned cattle, goat, sheep, giraffe, bison, yak, buffaloes, deer, camel, alpaca, llama, and antelope.

The forms of animal feed compositions include mash, pellets, crumbles, fines, flakes, pellets & flakes, mash & flakes, granules, or the like.

For example, the animal feed composition of the present invention can further comprises any material, such as corn gluten feed, sunflower hulls, distillers grains, guar hulls, wheat middlings, rice hulls, rice bran, oilseed meals, dried blood meal, animal by-product meal, fish by-product, fish meal, dried fish solubles, feather meal, poultry by-products, meat meal, bone meal, dried whey, soy protein concentrate, soy flour, yeast, wheat, oats, grain sorghums, corn feed meal, rye, corn, barley, aspirated grain fractions, brewers dried grains, corn flour, corn gluten meal, feeding oat meal, sorghum grain flour, wheat mill run, wheat red dog, hominy feed, wheat flour, wheat bran, wheat germ meal, oat groats, rye middlings, cotyledon fiber, ground grains, or a mixture thereof, in addition to β-alanine. In the case of ruminants, as the coarse feed, grass, wild grass, straw, stems and leaves of trees, rice straw, wheat straw, rice husks, soybean hulls, sawdust, bagasse, or the like may be included, as the concentrated feed, hull and berries such as corn, barley, rye, millet, cottonseed, soybean husks, rice bran, bran, or the like, rice brans such as rice bran, bran, or the like, oil meals such as soybean meal, rapeseed meal, or the like, spent grains such as beer meal, sake lees, soy sauce lees, or the like, fish meal, bone meal, or the like, can be included.

Feed additives can be included, in order to prevent feed quality deterioration, to supply nutrients and other active ingredients in feeds, and to promote the effective utilization of nutrients contained in feeds. For the object of the prevention of feed quality deterioration, there may be mentioned antioxidant, antifungal agent, binder, emulsifier, and conditioners.

For the purpose of supplementation of nutrients and other active ingredients in feeds, there may be mentioned amino acids, vitamins, minerals, and color enhancers (carotenoids). For the purpose of promoting the effective utilization of nutrients contained in feedstuffs, there may be mentioned antibacterial agents, antibiotics, flavoring agents, flavor enhancers, enzymes, bacteriostatic agent, and organic acids.

### <<Method for Increasing Muscle>>

The method for increasing muscle of the present invention comprises a step of administrating an effective amount of β-alanine or a salt thereof to a subject. That is to say, said β-alanine can be used in the method for increasing muscle. It is possible to increase muscle by administering an effective amount of β-alanine or its salt to a human or animal. The method for increasing muscle of the present invention may be performed when the subject is healthy, or when the subject is suffering from some disease. In other words, the method may be performed as a medical practice, or it may be performed outside of the medical practice.

When the subject is an animal, the method can be performed as a feeding method. It is possible to increase muscle by administering an effective amount of the composition for increasing muscle to humans or animals.

### <<β-Alanine for Use of Method for Increasing Muscle>>

Said β-alanine can be used for the method for increasing muscle. That is to say, the present specification discloses β-alanine for use of method for increasing muscle.

### <<Use of β-Alanine for Manufacturing Composition for Increasing Muscle>>

Said β-alanine can be used for manufacturing the composition for increasing muscle. That is to say, the present specification discloses a use of β-alanine for manufacturing the composition for increasing muscle.

### <<Functions>>

In the composition for increasing muscle of the present invention, the mechanism by which muscle can be increased has not been fully elucidated, but may be presumed to be as follows.

Skeletal muscle tissue contains stem cells called satellite cells. When the satellite cells are activated, they become progenitor cells called myoblasts.

Myoblasts differentiate into myocytes after proliferating through several cell divisions. Multiple myocytes fuse with each other to form multinucleated myotube cells.

The composition for increasing muscle of the present invention is considered to promote the differentiation of myoblasts into slow muscle cells (slow myocytes) and fast muscle cells (fast myocytes) and to increase the number of myotube cells, when myoblasts differentiate into myotube cells. Therefore, it is presumed that the composition for increasing muscle can promote the increase of slow muscle and fast muscle.

### EXAMPLES

The present invention will now be further illustrated by, but is by no means limited to, the following Examples.

### Example 1

In this example, β-alanine was added to C2C12 cells and the expression of MyHCI (slow muscle marker), MyHCIIa (fast muscle marker), MyHCIIb (fast muscle marker), and MyHCIIx (fast muscle marker) genes were examined.

### (1) Preparation of Total RNA

High Pure RNA Isolation Kit (Roche, Basel, Switzerland) was used for total RNA preparation. C2C12 cells were seeded at a concentration of 5.0×10⁴ cells/mL in 5 mL Dish. After 24 hours of incubation, β-alanine was added. 1×PBS was added to the control cells at the same volume as the added amount of β-alanine. Twenty-four hours after the addition of 3-alanine, sample was secondary added. After 24 hours of incubation, the culture supernatant was aspirated off. After washing the cells twice with 1×PBS, 1×PBS 200 μL/dish and 400 L/dish of Lysis/-binding buffer included in High Pure RNA Isolation Kit were added to the entire dish, and the total amount was collected in a 1.5-mL tube. The collected samples were suspended using a vortex mixer for 60 seconds. The filter tube and the collection tube in the kit were assembled, the sample solution was added to the filter tube, and centrifuged at 4° C., 9200×g for 15 seconds. The liquid drained into the collection tube was discarded, and the filter tube and collection tube were assembled again. In a 1.5-mL tube, 90 μL of DNase Incubation Buffer per sample and 10 μL of DNase I per sample were mixed. The mixture was added to the filter tubes and incubated at room temperature for 15 minutes. After incubation, Wash buffer I (500 μL) was added to the filter tubes and centrifuged at 4° C., 9200×g for 15 seconds. The liquid drained into the collection tube was discarded, and the filter tube and the collection tube were reassembled. Wash buffer II (500 μL) was added to the filter tube and centrifuged at 4° C., 9200×g for 15 seconds. The liquid drained into the collection tube was discarded, and the filter tube and the collection tube were reassembled. Wash buffer II (200 L) was added to the filter tube and centrifuged at 4° C., 13000×g for 2 minutes. The filter tube was inserted into a new 1.5-mL tube, and Elution buffer (70 μL) was added to the filter tube. The filter tube was left at room temperature for 3 minutes, and centrifuged at 4° C., 9200×g for 1 minute. The eluate obtained by the above procedure was used as the RNA solution. The concentration of RNA in the solution was calculated based on the absorbance value at 260 nm using a Nano Drop 2000/2000c spectrophotometer (Thermo Scientific, Waltham, MA, USA) and used in the subsequent experiments

### (2) Reverse Transcriptase Quantitative PCR (RT-qPCR)

The GoTaq1-StepRT-qPCR System (Promega, Madison, WI, USA) was used for RT-qPCR. In a 1.5-mL tube, 10 μL of GoTaq qPCR Master Mix per well, 0.4 μL of GoScriptRTMixfor1-StepRT-qPCR per well, and 1.6 μL of Nuclease-FreeWater per well were gently suspended on ice, to prepare a Reaction Mix. 12 μL of Reaction Mix, 2 μL of each of Forward/Reverse primers diluted to 2 μM with Nuclease-FreeWater, and 4 μL of RNA solution diluted with Nuclease-FreeWater were added to one well of a 96-well PCR plate (Nippon genetics, Tokyo, Japan), on ice. After the plated being centrifuged for 1 minute, it was set on the Thermal Cycle Dicer Real Time System (TaKaRaBio, shiga, Japan) and subjected to RT-qPCR. The reaction conditions were as follows: 1 cycle of 37° C. for 15 min, 1 cycle of 95° C. for 10 min, 45 cycles of 95° C. for 10 sec-60° C. for 30 sec-72° C. for 30 sec, and 1 cycle of 95° C. for 15 sec-60° C. for 30 sec-95° C. for 15 sec. Then, it was detected by FAM.

The expression levels of genes of interest were quantified relatively using the ΔΔCt method.

Primers were as follows.

MyHCI
Sense primer:
(SEQ ID NO: 1)
AGCATTCTCCTGCTGTTTCCT
Anti-sense primer:
(SEQ ID NO: 2)
GGCTGAGCCTTGGATTCTCA
MyHCIIa
Sense primer:
(SEQ ID NO: 3)
ATTCTCAGGCTTCAGGATTTGGTG
Anti-sense primer:
(SEQ ID NO: 4)
CTTGCGGAACTTGGATAGATTTGTG
MyHCIIb
Sense primer:
SEQ ID NO: 5)
CTGCAGGACTTGGTGGACAAACTA
Anti-sense primer:
(SEQ ID NO: 6)
TTGGCCAGGTTGACATTGGA
MyHCIIx
Sense primer:
(SEQ ID NO: 7)
CATCCCTAAGGCAGGCTCT
Anti-sense primer:
(SEQ ID NO: 8)
AGCCTCGATTCGCTCCTTTT

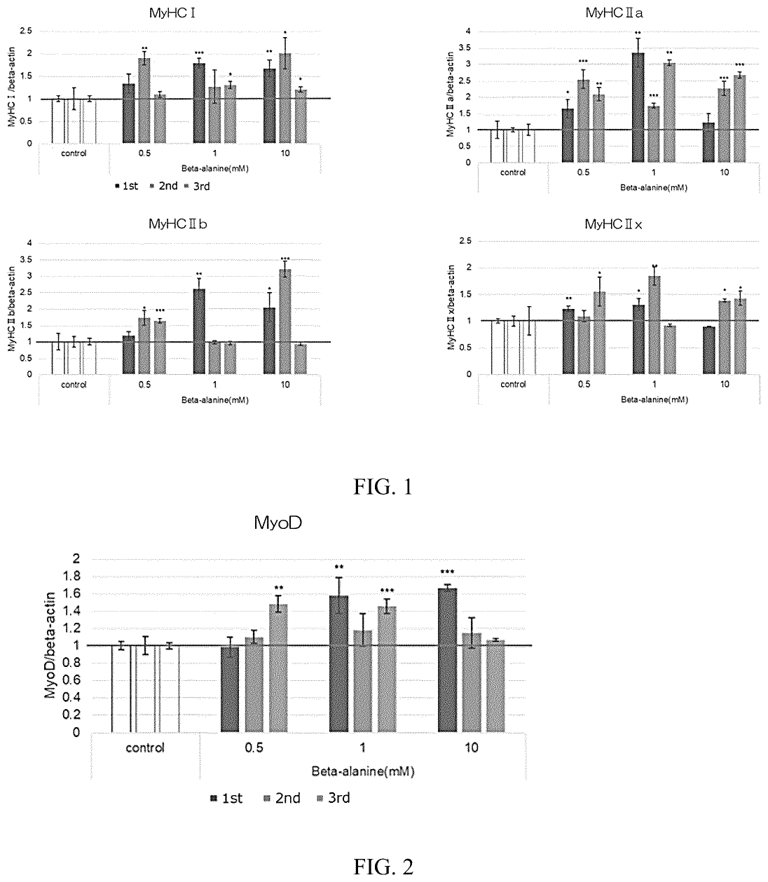

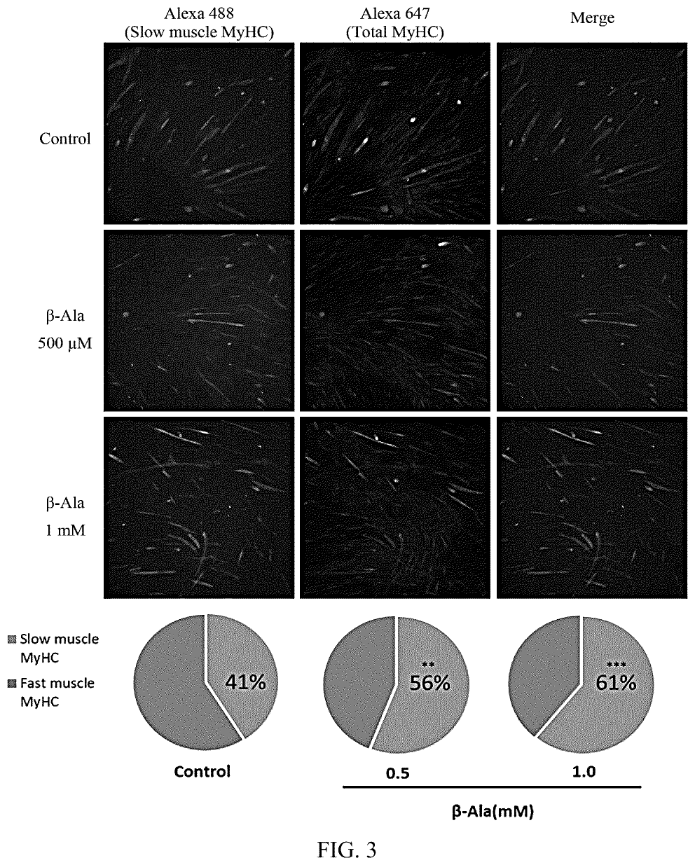

As shown in FIG. 1, the addition of β-alanine increased each expression of the slow muscle marker, MyHCI and the fast muscle markers, MyHCIIa, MyHCIIb, and MyHCIIx.

### Example 2

In this example, β-alanine was added to C2C12 cells and the expression of MyoD gene was examined.

The procedure described in Example 1 was repeated except that the following sense primer and antisense primer of MyoD were used.

MyoD
Sense primer:
(SEQ ID NO: 9)
ATGAGGCCTTCGAGACGCTC
Anti-sense primer:
(SEQ ID NO: 10)
CAGAGCCTGCAGACCTTCGA

As shown in FIG. 2, the addition of β-alanine increased the expression of MyoD, a transcriptional regulator of muscle differentiation that promotes differentiation from myoblasts to myotube cells.

### Example 3

In this example, the increase of differentiation of myoblasts into slow muscle cells was examined by the addition of β-alanine.

C2C12 cells were seeded onto Clear Fluorescence Black Plate (Greiner bio-one, Tokyo, Japan) at a concentration of 1.0×105 cells/mL, and after 48 hours, the medium was replaced with DMEM medium containing 2% Horse Serum (HS)(Thermo Fisher Scientific), to induce differentiation. Then, the medium was changed every 2 days and β-alanine (0.5 mM or 1.0 mM) was added every 2 days, and the following immunostaining was performed after 9 days of incubation from the induction of differentiation. For controls, 1×PBS in a volume equal to the amount of β-alanine added. To the cell culture medium, 100 μL/well of 8% paraformaldehyde solution was added, and the cells were fixed by incubation at room temperature for 15 minutes. After removing the culture supernatant and cell-fixing solution together, 100 μL/well of 1×PBS was added and allowed to stand for 5 minutes, and then it was removed. The cells were washed three times by repeating the above procedure. After washing, 100 μL/well of blocking buffer (1×PBS (10 mL), normal goat serum (0.50 mL), TritonX-100 (30 μL)) was added, and the cells were allowed to stand at room temperature for 1 hour. The blocking buffer was removed, and then 100 μL/well of primary antibody reaction solution was added, and the reaction was carried out overnight at 4° C. in the dark. As the primary antibody, a myotube-specific antibody (Anti-Fast Myosin Skeletal Heavy chain, Abcam; 1 μg/mL) or slow muscle-specific antibody (Anti-Slow Skeletal Myosin Heavy chain, Abcam; 1.25 μg/mL) was used. After overnight reaction, the cells were washed with 1×PBS for 5 min, 3 times, and 100 μL/well of the secondary antibody reaction solution was added, and the cells were allowed to react for 1 hour at room temperature in the dark. As the secondary antibody, Goat anti-Rabbit Alexa Fluor 647 (abcam; 2 μg/mL) or Goat anti-Mouse Alexa Fluor 488 (Jackson Immuno Research; 0.85 μg/mL) was used. After the reaction, the cells were washed with 1×PBS for 5 min, 3 times, and 100 μL/well of nuclear staining solution was added, and the cells were allowed to react for 20 min at room temperature in the dark. After the reaction, the cells were washed twice with 1×PBS, 150 μL/well of 1×PBS was added, and images were acquired by IN Cell Analyzer 2200 (GE Healthcare Japan, Tokyo, Japan). The images were analyzed by IN Cell Investigator High-content image analysis software (GE Healthcare Japan), and the area of Alexa Fluor 488-positive area (slow muscle) was divided by the area of Alexa Fluor 647-positive area (myotubes).

The percentage of slow muscle in the total myotubes was determined. The protocol “Hoechst_488_647” was used.

### Example 4

In this example, β-alanine was added to C2C12 cells and the expression of MyHCI (slow muscle marker), MyHCIIa (fast muscle marker), MyHCIIb (fast muscle marker), and MyHCIIx (fast muscle marker) genes were examined.

C2C12 cells were seeded in 6-well plates at a concentration of 2.0×10⁵ cells/mL, and after 48 hours the medium was replaced with DMEM medium containing 2% Horse Serum (HS) (Thermo Fisher Scientific) to induce differentiation. Further, after 24 hours, the medium was replaced with DMEM medium containing 2% HS. The medium was changed every 2 days thereafter, and further the medium changes and the additions of β-alanine (0.5 mM, 1 mM, or 10 mM) were conducted on the 8th and 9th day after seeding. The cells on 10th day after seeding were used for the experiments. For controls, 1×PBS was added at the same volume as the amount of β-alanine.

Total RNA preparation and RT-qPCR were performed according to Example 1.

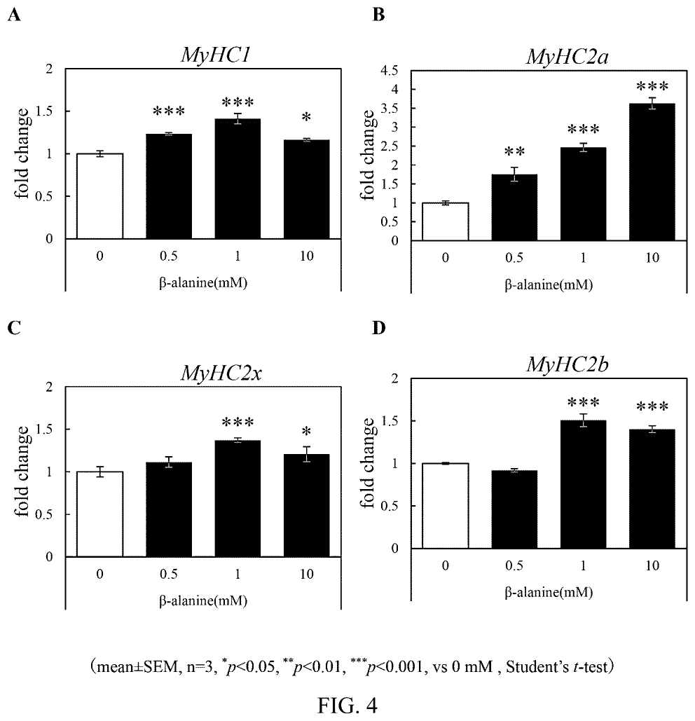

As shown in FIG. 4, in C2C12 cells wherein β-alanine was added thereto after differentiation, slow muscle marker, MyHCI was significantly increased at 0.5 mM, 1 mM, and 10 mM, and fast muscle marker, MyHCIIa also showed a concentration-dependent enhancement of gene expression at 0.5 mM, 1 mM, and 10 mM. In addition, MyHCIIx and MyHCIIb were significantly increased at 1 mM and 10 mM.

The addition of β-alanine promoted the differentiation of myoblasts into myotube cells and the number of myotubular cells were increased. In addition, the ratio of slow muscle cells increased as the amount of β-alanine increased. β-alanine promotes the increase of fast muscle cells and slow muscle cells, and in particular it is considered that the ratio of slow-muscle cells increases with increasing β-alanine concentration.

### INDUSTRIAL APPLICABILITY

The composition for increasing muscle of the present invention can be used as a pharmaceutical composition, a food composition, or a feed composition, and can increase muscle.

1. A composition for increasing muscle, comprising β-alanine or a salt thereof as an active ingredient.

2. The composition for increasing muscle according to claim 1, wherein the muscle is a slow muscle or a fast muscle.

3. The composition for increasing muscle according to claim 2, wherein muscle gain is a slow muscle gain or a fast muscle gain at differentiation of myoblasts into myotube cells.

4. The composition for increasing muscle according to claim 1, wherein the composition is food composition.

5. A method for increasing muscle, comprising a step of administrating an effective amount of β-alanine or a salt thereof to a subject.

6. The method for increasing muscle according to claim 5, wherein the muscle is a slow muscle or a fast muscle.

7. The composition for increasing muscle according to claim 2, wherein the composition is food composition.

8. The composition for increasing muscle according to claim 3, wherein the composition is food composition.


SEQUENCE LISTING (10 sequences)

SEQ ID NO: 1 - DNA, 21 bp, synthetic construct
  source 1..21: mol_type = other DNA
agcattctcctgctgtttcct

SEQ ID NO: 2 - DNA, 20 bp, synthetic construct
  source 1..20: mol_type = other DNA
ggctgagccttggattctca

SEQ ID NO: 3 - DNA, 24 bp, synthetic construct
  source 1..24: mol_type = other DNA
attctcaggcttcaggatttggtg

SEQ ID NO: 4 - DNA, 25 bp, synthetic construct
  source 1..25: mol_type = other DNA
cttgcggaacttggatagatttgtg

SEQ ID NO: 5 - DNA, 24 bp, synthetic construct
  source 1..24: mol_type = other DNA
ctgcaggacttggtggacaaacta

SEQ ID NO: 6 - DNA, 20 bp, synthetic construct
  source 1..20: mol_type = other DNA
ttggccaggttgacattgga

SEQ ID NO: 7 - DNA, 19 bp, synthetic construct
  source 1..19: mol_type = other DNA
catccctaaggcaggctct

SEQ ID NO: 8 - DNA, 20 bp, synthetic construct
  source 1..20: mol_type = other DNA
agcctcgattcgctcctttt

SEQ ID NO: 9 - DNA, 20 bp, synthetic construct
  source 1..20: mol_type = other DNA
atgaggccttcgagacgctc

SEQ ID NO: 10 - DNA, 20 bp, synthetic construct
  source 1..20: mol_type = other DNA
cagagcctgcagaccttcga